(19) United States
(12) Patent Application Publication
(10) Pub. No.: US 2003/0032936 A1
(43) Pub. Date: Feb. 13, 2003

(54) Side-exit catheter and method for its use

(76) Inventor: Robert J. Lederman, Chevy Chase, MD (US)

Correspondence Address: KLARQUIST SPARKMAN, LLP, One World Trade Center, Suite 1600, 121 S.W. Salmon Street, Portland, OR, 97204, US

(21) Appl. No.: 09/927,603

(22) Filed: Aug. 10, 2001

(51) Int. Cl. A61M 31/00

(52) U.S. Cl. 604/507; 607/120; 607/123; 604/164.09

(57) ABSTRACT

Disclosed is a device for delivering a therapeutic or diagnostic agent to an anatomic position, such as a heart. The device includes a flexible catheter having a proximal end and a distal end, a guide wire lumen that extends longitudinally through the catheter, and a delivery lumen that extends longitudinally at least partially through the catheter. The delivery lumen communicates with a side port adjacent a distal end of the catheter, through which side port a therapeutic or diagnostic agent may be delivered. The device also may include a guide wire, and the catheter may be advanced along the guide wire via the guide wire lumen. The device also may include a secondary catheter capable of sliding through the delivery lumen and capable of delivering a liquid, solid, or radiant agent to a desired anatomic location; thus, the secondary catheter may be considered a delivery catheter. The device may be used to deliver a therapeutic agent to a particular body location in a subject suffering a disease. In certain embodiments, the device may be used to deliver an ablative agent to the heart of a subject suffering hypertrophic cardiomyopathy.

### FIELD

This invention relates to medical devices for delivering therapeutic and diagnostic agents to particular regions of the body, including catheters for delivering such agents into the heart, and methods for their use.

### BACKGROUND

Heart tissue (myocardium) may be altered by delivering drugs, electromagnetic energy, or mechanical force to the myocardium through the inner layer of the heart (the endocardium) via a process called “endomyocardial delivery.” In some cases of heart disease, enlargement or excess growth of myocardium obstructs the flow of blood from the heart. For example, in the heart disease “hypertrophic cardiomyopathy,” a region of heart tissue dividing the two ventricles located beneath the aortic valve is sometimes enlarged and obstructs blood flow from the left ventricle into the aorta.

Treatments for such cases of heart disease often include the selective surgical removal of the excess heart tissue, a procedure called “septal myotomy-myectomy.” However, a different therapeutic treatment has been proposed that includes selectively and intentionally damaging excess myocardium using a drug (such as ethanol or phenol), laser energy, or electromagnetic radiation (such as laser or radiofrequency energy) to locally reduce the heart wall thickness. Reducing the thickness of the myocardium consequently improves the flow of blood from the heart.

Concentric lumen catheters can be used to deliver a needle or ablative energy to the endomyocardium. Such catheters are introduced into the left ventricle through the aorta and aortic valve (located near the top of the left ventricle) and are inherently directed along their path of entry to the bottom (base) of the heart. It is difficult-and often mechanically impossible-for a catheter to “turn around” on itself and treat areas of the myocardium immediately underneath the aortic valve. Specifically, bulky or retroflexed catheters may not fit within the ventricular cavity of patients with hypertrophic cardiomyopathy or may induce ventricular arrhythmias by mechanically stimulating the endocardial wall by virtue of their bulk. While some concentric catheters are steerable and can be directed against a side wall of the heart, these catheters are still limited in their ability to make the “U-turn” required to treat the ventricular septum underneath the aortic valve.

For example, U.S. Pat. No. 6,053,911 discloses a catheter for use in transmyocardial revascularization (TMR). This procedure involves introducing the catheter into a coronary artery (one of the vessels that supplies blood to the heart) and forming a channel from the blood vessel into the heart muscle, which helps oxygenate hypoxic heart tissue. The catheter has an internal lumen that distally curves gently to form a deflecting surface that directs a channel forming catheter out of the side of the catheter. This directs the channel forming catheter substantially perpendicularly out of the coronary artery and into the myocardial wall.

As another example, U.S. Pat. No. 6,126,654 and PCT Publication WO 99/17671 also disclose a TMR catheter that forms channels in the wall of the heart, but this catheter is introduced into the ventricle instead of the coronary arteries. The catheters in both of these references have a steerable distal end that can be directed at a specific area of the endocardium. However, these catheters lack the flexibility to turn back on themselves and treat the area under the aortic valve.

As another example, PCT publication WO 96/35469 discloses a steerable concentric lumen catheter having a distal end that can be directed at selected areas of the myocardium. This catheter, like the others, cannot be used to treat the internal wall of the heart immediately below the valve through which the catheter is introduced into the ventricle because the distal end of the catheter cannot be turned back on itself tightly enough.

Unfortunately, it is this area of the ventricular septum beneath the aortic valve (the “endomyocardial septum”) that is targeted for ablation in the treatment of hypertrophic cardiomyopathy.

### SUMMARY

Disclosed is a device or assembly for delivering a therapeutic or diagnostic agent to an anatomic position within a human or animal body, such as a heart. The device includes a flexible catheter having a proximal end and a distal end, a guide wire lumen that extends longitudinally through the catheter, and a delivery lumen that extends longitudinally at least partially through the catheter. The guide wire lumen and delivery lumen may be separate from one another and may extend substantially parallel to one another through the catheter. If the guide wire lumen extends completely through the catheter, the guide wire lumen defines guide wire ports in each end of the catheter.

The delivery lumen communicates with a side port adjacent an end of the catheter, through which side port a therapeutic or diagnostic agent may be delivered. In some embodiments, the delivery lumen includes a deflection member, such as an intermediate curved wall or shoulder, that connects the longitudinally extending delivery lumen with the side port that opens through a side wall of the catheter. Thus, the curved wall or shoulder changes the direction in which the delivery lumen extends from longitudinally through the catheter to substantially transverse to the longitudinal axis of the catheter. The device also may include a secondary catheter capable of sliding through the delivery lumen.

The catheter may be a steerable catheter or a guideable catheter. In some embodiments, a guideable catheter includes a guide wire along which the guide wire lumen is capable of sliding. Thus, the guide wire allows an operator to guide the catheter into a desired anatomic position, such as a chamber or structure within the heart.

The delivery lumen, or a secondary catheter introduced through the delivery lumen, is capable of delivering a pharmaceutical, chemical, biological, mechanical, or radiant energy agent, as a solid, liquid, gas, or radiation, to a desired anatomic location. The secondary catheter may be considered a delivery catheter. In specific embodiments, the secondary catheter includes an injection needle for delivering a therapeutic agent, such as a pharmaceutical agent; an ablative agent, such as a caustic chemical; or cells for cellular transfer. In other specific embodiments, the secondary catheter includes a laser, a radiofrequency probe, or a cryogenic probe.

The device may be sold as part of a kit that includes a catheter, as described above, and a guide wire along which the guide wire lumen of the catheter can slide. The kit also may include an agent delivery device for introducing a therapeutic or diagnostic agent through the delivery lumen. In some embodiments, the agent delivery device is a flexible secondary catheter as described above. In specific embodiments, the agent delivery device is a flexible secondary catheter that includes an energy delivery probe, such as a radiofrequency, mechanical, or thermal energy delivery probe. The kit also may include instructions for advancing the catheter along the guide wire to a desired anatomic location, such as into a ventricle of the heart, and delivering a therapeutic or diagnostic agent through the delivery lumen and side port to that location. For example, the instructions may describe how to advance the catheter along the guide wire through the vasculature into a ventricle of the heart, such that the side port is positioned adjacent a ventricular septum, and then delivering an agent through the delivery lumen to the ventricular septum.

Also disclosed is a method of delivering a therapeutic or diagnostic agent to a desired anatomic location, such as a heart, using the device described above. In certain embodiments, the elongated flexible catheter is provided and advanced into a chamber of a heart until the side port is substantially aligned with a target structure in the chamber of the heart. Once the side port is properly aligned, a therapeutic or diagnostic agent is introduced through the delivery lumen and side port and directed at the target structure. The catheter may be advanced into the heart by advancing the guide wire lumen of the catheter along an intra-vascular guide wire that extends into the heart chamber. The intra-vascular guide wire may be introduced percutaneously into a blood vessel and then advanced through the vasculature into the chamber of the heart. The target structure of the heart may be a ventricular septum, such as an area of the septum of the left ventricle beneath the aortic or mitral valve. However, other areas of the heart, such as a different region of the endocardium, may be targeted.

In some embodiments, the device is used to ablate tissue in a subject suffering some disease. In more specific embodiments, the device is used to ablate tissue in the aortic outflow tract of a heart in a subject who has hypertrophic cardiomyopathy. For example, thermal or radiofrequency energy may be delivered through the delivery lumen and through the side port into the endocardium of a heart. In these embodiments, the agent introduced through the delivery lumen is an ablative agent. In other non-limiting examples, the agent is a pharmaceutical, chemical, biological, mechanical, or radiant energy agent that may or may not be an ablative agent. In some particular embodiments, however, the pharmaceutical, chemical, biological, mechanical, or radiant energy agent is an ablative agent.

### BRIEF DESCRIPTION OF THE DRAWINGS

FIG. 1 is a fragmentary external, side elevation view of one embodiment of the device, with a guidewire extending through it.

FIG. 2 is an enlarged longitudinal section of a distal end of the device shown in FIG. 1.

FIG. 3 is an enlarged view of the distal tip of the device shown in FIG. 2, but with a secondary catheter, having a needle, extending through the delivery lumen.

FIG. 4 is an enlarged view of the distal tip of the device shown in FIG. 2, but with a secondary catheter, having a laser ablation device, extending through the delivery lumen.

FIG. 5 is an enlarged view of the distal tip of the device shown in FIG. 2, but with a liquid agent being delivered through the delivery lumen.

FIG. 6 is an anterior view of a heart in partial cross section illustrating one method for introducing the catheter into the left ventricle of the heart.

FIG. 7 is an enlarged view of the encircled area of FIG. 6 with the pulmonary artery and surrounding tissue removed to show the distal end of device disposed within the aortic valve.

### DETAILED DESCRIPTION

As used herein, the singular forms “a,” “an,” and “the,” refer to both the singular as well as plural, unless the context clearly indicates otherwise. For example, the term “a delivery lumen” includes single or plural lumens and can be considered equivalent to the >—phrase “at least one delivery lumen.”

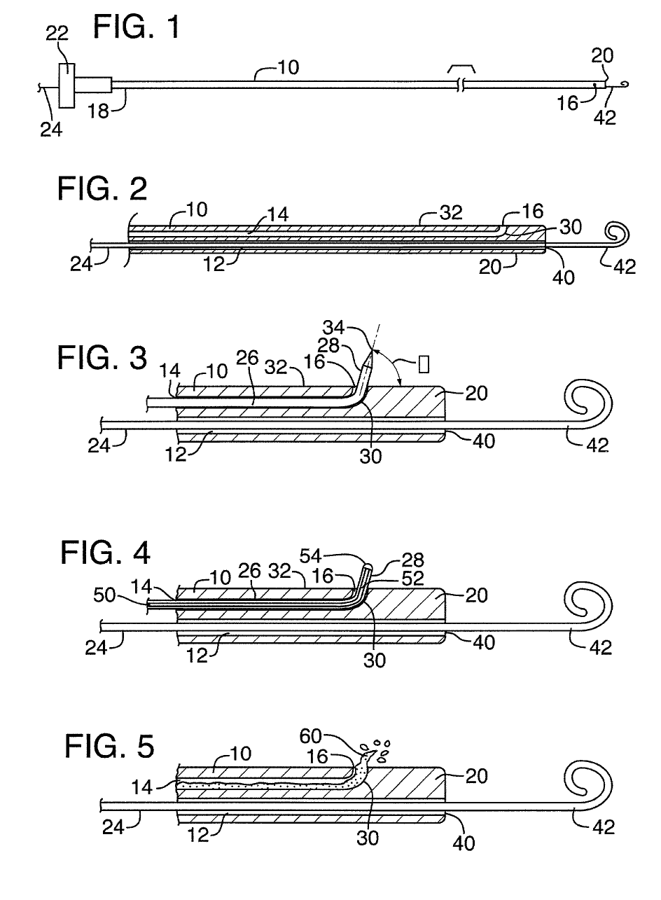

A device for delivering a therapeutic or diagnostic agent to a heart is disclosed. In the embodiment illustrated in FIGS. 1-5, the device includes an elongated, flexible two-lumen cathether 10, a guide wire lumen 12, and a delivery lumen 14. Guide wire lumen 12 extends longitudinally through catheter 10, and delivery lumen 14 extends longitudinally at least partially through catheter 10. As illustrated in FIGS. 2-5, these lumens 12, 14 are separate and extend substantially parallel to one another through catheter 10, and delivery lumen 14 communicates with side port 16 disposed along catheter 10. While the illustrated embodiment includes two separate lumens 12, 14 running substantially parallel to each other, alternative embodiments of the device contain a single lumen that serves as both the guide wire lumen and delivery lumen. Alternatively, the device has two lumens that do not run substantially parallel to each other, or the lumens are concentric (for example, with a central guide lumen extending through an annular delivery lumen). In other embodiments, the guide wire lumen is replaced by an external set of guide wire guides that provide a “monorail” system. In such embodiments, the guide wire is inserted through a plurality of guide wire guides mounted on an exterior surface of the flexible catheter. The guide wire guides may be any size or suitable shape, for example, a plurality of semi-circular elements defining aperatures through which the guide wire is inserted.

As illustrated in FIG. 1, catheter 10 has a proximal end 18 and a rounded distal end 20, with a guide collar 22 coupled to proximal end 18 of catheter 10. Various control mechanisms, including electrical, optical, or mechanical control mechanisms, may be attached to catheter 10 via guide collar 22. For example, in the illustrated embodiments, guide wire 24 is included as a mechanical control mechanism. The guide collar may include additional operational features, such as a grip for aiding manual control of the catheter, markers indicating the orientation of the delivery lumen or guide wire lumen, markers to gauge the depth of delivery lumen advancement, instruments to measure catheter operation, such as a temperature guage to monitor RF or cryogenic ablation, or an injector control mechanism coupled to the delivery lumen for delivering a small, precise volume of injectate. In some embodiments, the guide collar contains instrumentation electrically coupled to metallic braiding within the catheter, thus allowing the catheter to simultaneously be used as a receiver coil for magnetic resonance imaging (MRI).

In the illustrated embodiment, guide wire 24 has been inserted into guide wire lumen 12 via guide collar 22 and can be seen emerging from the distal end 20 of catheter 10. As illustrated, the distal end 42 of guide wire 24 optionally may be coiled to form a “pigtail.”

As illustrated in FIG. 3, secondary catheter 26 has been inserted into delivery lumen 14, and the distal end 28 of secondary cathether 26 is seen emerging from side port 16 at angle θ of about 75°. Delivery lumen 14 and side port 16 may be oriented to change angle θ to an angle of about 10° to about 170°. In particular embodiments, angle θ is from about 30° to about 150°, such as about 45° to about 135°, or more particularly, from about 60° to about 120°. In alternative embodiments, angle θ is from about 30° to about 90°, or more particularly, about 90°. In this illustrated embodiment, the agent delivered through secondary catheter 26 also is delivered through side port 16.

As illustrated in FIGS. 2-5, delivery lumen 14 includes a deflection member 30 connecting it to side port 16. As illustrated, deflection member 30 is an intermediate curved wall at a terminal end of delivery lumen 14, adjacent the distal end 20 of catheter 10, that connects delivery lumen 14 to side port 16. Thus, deflection member 30 may be considered a shoulder portion of delivery lumen 14 that curves from a longitudinal to a substantially transverse direction within flexible catheter 10. When inserted through longitudinally extending delivery lumen 14, the distal end 28 of secondary catheter 26 contacts this deflection member 30 and is deflected through side port 16. Thus, in the illustrated embodiment, delivery lumen 14 extends longitudinally through the catheter 10, curves to a direction substantially transverse the longitudinal axis of catheter 10 at deflection member 30 near the distal end 20 of catheter 10, and communicates with side port 16 that opens through a side wall 32 of the catheter 10.

The deflection member may be shaped or formed as a structure other than a curved wall or shoulder, so long as the deflection member deflects the secondary catheter from the delivery lumen to the side port. For example, rather than being a curved shoulder portion, the deflection member may be a strut extending at an angle to the longitudinal path of delivery lumen 14, to divert the secondary catheter toward side port 16.

In some embodiments, the delivery lumen extends along the flexible catheter beyond the side port to nearer the distal end of the catheter. In particular embodiments, which are not illustrated in the figures, the delivery lumen extends through both the proximal end and distal end of the catheter. In these embodiments, the deflection member is placed in an intermediate position within the delivery lumen, rather than at the end of the delivery lumen, and may be removable. If a removable deflection member is used, two alternate pathways for the secondary catheter are provided within the delivery lumen-deflection through the side port, or extension within the delivery lumen beyond the side port to or through the distal end of the flexible catheter.

The catheter and/or secondary catheter may be composed of any material, or combination of materials, providing the strength and flexibility suitable to resist collapse by external forces, or forces imposed during bending or twisting. Exemplary materials include, but are not limited to: polymers, such as polyethylene or polyurethane; carbon fiber; or non-ferromagnetic metals, such as Nitinol, platinum, titanium, tungsten, copper, or nickel. The catheter and/or secondary catheter optionally may be reinforced with fibers of metal, carbon fiber, glass, fiberglass, a rigid polymer, or other high-strength material.

The guide wire may be composed of any material having the strength and flexibility suitable for use with the device. Suitable materials include a strand of metal, such as surgical stainless steel, Nitinol, platinum, titanium, tungsten, copper, or nickel; carbon fiber; or a polymer, such as braided nylon.

The dimensions of the device will depend on the characteristics of the subject treated and the methods used. In some embodiments, the catheter is about 50 to 200 cm long and about 2 to 30 mm in diameter. In particular embodiments, the catheter is about 80 to 100 cm long and about 2 to 4 mm in diameter. For example, a catheter of about 130 to 150 cm in length with a diameter of about 3 mm may be introduced into the femoral artery in the thigh of an adult human patient and guided into the left ventricle of the heart. The guide wire is dimensioned to operate with the catheter and is usually longer than the catheter. For example, a guide wire of about 100 to about 250 centimeters in length and about 0.1 to 2 mm in diameter may be used with the catheter described above. The secondary catheter also is dimensioned to operate with the catheter and is usually longer than the catheter. For example, a secondary catheter of about 100 to about 250 centimeters long and about 1 to 10 mm in diameter may be used with the catheter described above.

While the device described immediately above is dimensioned for introduction into the femoral artery in the thigh of an adult human patient and guiding into the left ventricle of the heart, devices for other uses and/or for other subjects may be sized differently. For example, a device introduced into the brachial or radial artery of a human patient might be shorter in length, and a device used with a dog might have a shorter length and smaller diameter. Additionally, the catheter, guide wire, and secondary catheter may be any shape in cross-section, though some embodiments employ catheters, guide wires, and secondary catheters that are round or elliptical in cross-section.

As illustrated in FIGS. 3-4, the diameter of guide wire lumen 12 is sized to receive guide wire 24, and the diameter of delivery lumen 14 is sized to receive secondary catheter 26. If snugly inserted into their respective lumens, the guide wire, secondary catheter, guide wire lumen, and/or delivery lumen may be coated with a substance, such as a Teflon® coating or a lubricant, to facilitate insertion or movement of the guide wire and/or secondary catheter.

The side port may be disposed anywhere along the catheter. In some embodiments, the side port is disposed adjacent the distal end of the catheter at a distance of from about 1 mm to about 50 cm proximal to the distal tip of the catheter. In particular embodiments, the side port is disposed from about 5 mm to about 25 cm proximal to the distal tip of the catheter, such as about 10 mm proximal to the distal tip of the catheter.

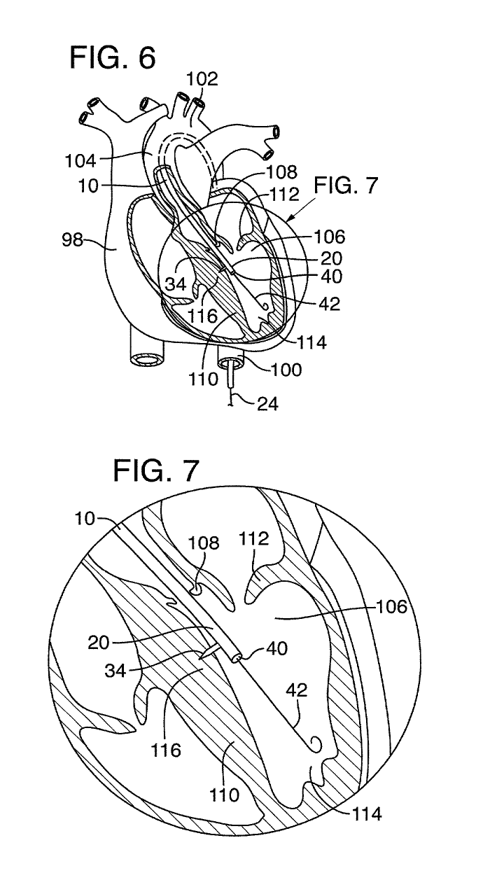

As illustrated, flexible catheter 10 is a guideable catheter. In this embodiment, guide wire 24 extends longitudinally completely through the catheter to form guide wire ports in each end of the catheter (only distal guide wire port 40 is shown in the drawings), and guide wire lumen 12 is capable of sliding along guide wire 24 to guide the distal end 20 of catheter 10 to a desired anatomic position within the subject. Thus, guideable catheter 10 is directed along guide wire 24 to a desired anatomic position. For example, as described in more detail below, FIGS. 6 and 7 illustrate the guiding and placement of catheter 10 within a heart 98.

In some embodiments, the distal end 42 of guide wire 24 is curved or coiled to form a “pigtail.” This pigtail provides an aid in guiding catheter 10 by providing a capture for catheter 10 as it slides along guide wire 24 via guide wire lumen 12. Additionally, the pigtail at the distal end 42 of guide wire 24 reduces the risk of guide wire 24 puncturing or damaging tissue within the body of a subject. For example, as illustrated in FIGS. 6-7, the distal end 42 of guide wire 24 is positioned near the wall of heart 98 at the base of left ventricle 106; if guide wire 24 is accidentally advanced into the heart wall, the pigtail reduces the likelihood that the distal end 42 of guide wire 24 would accidentally puncture the myocardial wall at or near the base of the left ventricle. A large, torqueable pigtail may be used to assist manipulation of the catheter into apposition with the target structure, such as the wall of a heart. A bend or kink within the pigtail distally may be used to assist such manipulation, and also a curve or kink in the catheter, with suitable transmission of torque.

In other embodiments, the flexible catheter is a steerable catheter, rather than a guideable catheter. In such embodiments, the distal end of the flexible catheter is actively steered to a desired anatomic position via some steering mechanism. Various steering mechanisms are known, such as those discussed in U.S. Pat. Nos. 6,126,654; 6,053,911; and 5,190,050. For example, the flexible catheter may contain one or more additional lumens holding pullwires that extend longitudinally through the catheter and are anchored near the distal tip of the catheter, with the proximal ends of the pullwires connected to suitable control mechanisms, such as pulling mechanisms. Each pullwire imparts an additional steering degree of freedom for steering the catheter.

The secondary catheter is capable of delivering a diagnostic or therapeutic agent to an anatomic location within the body of a subject, such as the heart. The agent may be a solid, liquid, gas, or radiation, and may be a pharmaceutical, chemical, biological, mechanical, or radiant energy agent. Suitable diagnostic and therapeutic agents include, but are not limited to, the particular agents disclosed herein.

Pharmaceutical agents include drugs commonly available to treat disease, such pain relievers, anti-cancer agents, antibiotics, anti-thrombotic agents, antivirals, and enzymatic inhibitors. Chemical agents include non-pharmaceutical chemicals, such as ethanol, phenol, chelating agents, and contrast agents for imaging particular structures of the body, including contrast agents for X-ray, fluoroscopy, ultrasound, computerized tomogrophy (CT), and MRI. Biological agents include nucleic acids, amino acids, cells, viruses, prions, biochemicals, vitamins, and hormones. Mechanical agents include mechanisms for monitoring, visualizing, or manipulating internal portions of a body, including thermometers and other sensors, cameras, probes, needles, knives, electrocautery snares, biopsy forceps, and suction tubes. Radiant energy agents include acoustic, thermal, and electromagnetic energies, such as infrared, ultraviolet, x-ray, microwave, radiofrequency, ultrasound, and laser. In some embodiments, plural agents are mixed or delivered together. As just one, non-limiting example, ethanol (an ablative agent) may be mixed with a contrast agent, such as microbubbles for sonographic contrast, iodinated radiocontrast for x-ray contrast, or a metal chelate for MRI contrast.

In certain embodiments, the agent or mechanism delivered by the secondary catheter is capable of ablating tissue; thus, the secondary catheter may include means for ablating tissue using one of the pharmaceutical, chemical, biological, mechanical or radiant energy agents described above. Suitable means for ablating tissue may be a needle or a mechanical cutting or boring apparatus, an optical fiber delivering laser energy, a chemical ablation device, a radiofrequency electrode delivering radiofrequency energy, or a cryogenically cooled probe. A number of means for ablating tissue have been described, such as in U.S. Pat. Nos. 6,053,911; 6,126,654; and 6,237,355; and in PCT Application WO 99/17671.

FIG. 3 illustrates a needle at the distal end 28 of secondary cathether 26. A needle is one, non-limiting example of a ablation device. A solid needle alone may be used to puncture and ablate tissue, or a hollow needle may be used to deliver an ablative agent, such as ethanol or phenol, to the tissue.

Other embodiments employ different ablation devices. For example, a mechanical cutting or boring apparatus may be made from a stiff wire having a sharp tip and sufficient column strength to be forced into the myocardium. Alternatively, a stainless steel or Nitinol hypotube may be used to punch into the myocardium. Additionally, a mechanical scraper, coring device, screw drive, drill bit, or biopsy forceps may be used to remove tissue. As another specific, non-limiting example, a mechanical cutting apparatus may be a device similar to a hole saw or open drill bit. With such a device, a bore is attached to a shaft, housed within the secondary catheter, by welding, adhesives or solder. This bore supports a radial cutting blade having cutting edges facing the direction of rotation connected by a guidewire mounting ring. The edge of the bore is sharpened or serrated, and a slightly pitched, rotating cutting core is displaced within the bore to drive cut myocardial tissue into the lumen of the secondary catheter.

Fiber optic lasers may be used to ablate tissue, and laser power of about 30 to 75 mJ/mm² can be used to ablate myocardial tissue. A laser beam may be produced by a device and directed into the proximal end of an optical fiber housed within the secondary catheter, and a lens may be mounted on the distal end of the same optical fiber for focusing or dispersing the laser beam as it emerges from the optical fiber, such as a simple telescope lens assembly. For example, as illustrated in FIG. 4, optical fiber 50 runs longitudinally through secondary catheter 26. The distal end 52 of optical fiber terminates at lens 54, and the proximal end (not shown) of optical fiber 50 is operably coupled to a laser source (not shown), such as a commercially available laser apparatus. Lens 54 may focus or diffract the laser energy emerging from optical fiber 50, depending on the needs of the operator, to facilitate tissue ablation. Commercially available laser-based systems include the Axcis™ laser catheter system from CardioGenesis Corporation (Foothill Ranch, Calif.), and the OmniPulse™ MAX surgical laser system available from Trimedyne, Inc. (Irvine, Calif.). Additionally, an excimer laser, available from Spectranetics Corporation (Colorado Springs, Colo.), produces a laser beam with a wavelength of 308 nm. Additionally, U.S. Pat. No. 5,728,091, describes a laser-based fiber optic probe for forming channels in a myocardium, which may be adapted for use in the secondary catheter.

The delivery lumen may be used to deliver an ablative agent (and/or a diagnostic or other therapeutic agent) to the distal end of the cathether. The agent is introduced into the delivery lumen, such as injected into the delivery lumen by a syringe or other injection apparatus, and the agent flows through the delivery lumen until it is directed laterally through the side port, for example toward a septal wall of the heart, as illustrated in FIG. 5. In the embodiment illustrated in FIG. 5, liquid ablative agent 60 has been introduced into the proximal end (not shown) of delivery lumen 14 and caused to flow through delivery lumen 14 and side port 16 into the space adjacent the distal end 20 of catheter 10. Optionally, an infusion port for selectively releasing the agent may be located at or near the side port. The infusion port stops the flow of the agent within the delivery lumen and allows the agent to be stored within the delivery lumen until the port is triggered to release the agent. The infusion port may be a valve or seal that automatically releases the agent when the agent is pressurized, or the infusion port may be mechanically or electronically coupled to some control mechanism at the proximal end of the catheter, such as a pull wire or trigger located on the guide collar. For example, the triggering mechanism disclosed in U.S. Pat. No. 6,254,573 may be adapted for use with the catheter. Additionally, in some embodiments, the catheter includes plural delivery lumens for delivering one or more agents, and release of the agent from the delivery lumens may be independently or collectively controlled.

In some embodiments, the ablative agent is delivered through the delivery lumen via a secondary catheter having at least one lumen extending longitudinally through it. In such embodiments, the secondary catheter is inserted into and advanced through the delivery lumen until the distal tip of the secondary catheter emerges from the side port. The agent is introduced into the lumen of the secondary catheter and directed out through the distal tip of the secondary catheter. Additionally, the secondary catheter optionally may contain an infusion port at its distal tip.

Once the distal end of the device is placed at a desired location, an ablative agent, such as ethanol or phenol, is injected through the delivery lumen—either through the delivery lumen itself or the secondary catheter lumen—and laterally into the space adjacent the distal end of the device.

Radiofrequency (RF) ablation devices also may be used as part of the secondary catheter. In such an embodiment, an RF electrode is mounted on the distal tip of the secondary catheter. The RF electrode is a conductor electrically connected to an RF generator through a electrical wire disposed within the secondary catheter. Suitable RF generators are commercially available, such as the Surgitron® model manufactured by Ellman International Inc. (Hewlett, N.Y.). The electrode may be operated in a monopolar mode, in which case a patch electrode will be placed on the skin of the subject to complete the electrical pathway required for the RF electrode. Alternatively, the secondary catheter may be provided with bipolar capability by placing a ground electrode in close proximity to the RF electrode, either on the secondary catheter or on the flexible catheter.

A cryogenic ablation device also may be used as part of the secondary catheter. In such an embodiment, a probe is caused to contact tissue and then cooled by a circulating refrigerant to a temperature low enough to cause ice crystals to form in the tissue. Intracellular ice crystals can induce cell lysis, thus leading to ablation of tissue. One such cryogenic probe is described in U.S. Pat. No. 6,237,355, and is commercially available as the Glacier™ Cardiac Ablation System from CryoGen, Inc. (San Diego, Calif.).

Similar to the flexible catheter, the secondary catheter may be guideable or steerable. A guideable secondary catheter may be inserted into the delivery lumen of the catheter and manually advanced through the delivery lumen. For example, with reference to FIG. 3, an operator could simply insert the distal end 28 of secondary cathether 26 into the proximal delivery port (not shown) of catheter 10 and manually push the length of secondary catheter 26 into cathether 10 until the distal end 28 of secondary catheter 26 emerges from the delivery lumen 14 and side port 16. However, the secondary catheter may be steerable if it includes some steering mechanism, such as the mechanisms described above with reference to the flexible catheter. For example, the secondary cathether may include a steerable distal tip for maneuvering that portion of the secondary catheter extending from the delivery lumen through the side port.

The device may be sold as part of a kit. In some embodiments, the kit includes a flexible catheter and a guide wire. In more specific embodiments, the kit also includes an agent delivery device, such as a secondary catheter, for introducing an agent through the delivery lumen to a desired anatomic location. Additionally, the kit may contain instructions for use, such as instructions for using the device for one or more of the uses described herein. For example, the instructions may describe advancing the catheter along the guide wire into a ventricle of the heart with the side port adjacent a ventricular septum of the heart, then delivering a therapeutic or diagnostic agent through the delivery lumen and side port to the ventricular septum.

The device may be used in a variety of ways, such as for delivering a therapeutic or diagnostic agent to a particular organ, tissue, interstitial space, or other anatomic location within a subject. In some embodiments, the device is used to deliver a therapeutic or diagnostic agent to the endocardium of a heart. In such embodiments, the distal end of the flexible catheter is advanced into a chamber of the heart until the side port is substantially aligned with a target position or structure within the heart chamber. Once aligned, the therapeutic or diagnostic agent is introduced into the heart through the delivery lumen and side port.

The flexible catheter may be guided into the heart by advancing the guide wire lumen of the catheter along an intra-vascular guide wire that extends into the heart. In such an embodiment, the guide wire is first introduced into the vascular system and guided into the heart. The guide wire may be introduced percutaneously, or through open surgery, into any blood vessel appropriate for guiding the guide wire into a target chamber or structure of the heart. In particular embodiments, the guide wire is introduced percutaneously into a femoral artery by a cutdown of the artery or via the Seldinger technique. In other particular embodiments, the guide wire is introduced percutaneously into the brachial artery in the arm of the subject, or into the right subclavian artery. In alternative embodiments, the catheter is introduced into a vein, such as the femoral or jugular vein, and guided into the right ventricle of the heart. In still other embodiments, the catheter is introduced into other vascular or perivascular structures, such as the liver, aorta, or a tumor.

However, the catheter may be introduced into the body in some other manner. For example, a steerable catheter may be introduced without the aid of the guide wire, or the guide wire and catheter—and, optionally, the secondary catheter—may be introduced into the body at the same time.

After insertion into a blood vessel, the guide wire is then advanced through the vasculature into the heart by progressive manual insertion. Advancing the guide wire may be aided by imaging enhancement. For example, the guide wire may include a radiopaque marker adjacent its distal end, such as a platinum or tantalum band around the circumference of the guide wire, for image enhancement using an imager, such as a fluoroscope or magnetic resonance imaging (MRI) system. As another example, a fiber optic scope may be used to visualize the position of the guide wire within the subject.

After insertion, the guide wire is advanced through the vasculature to the desired position within the heart of the subject. For example, as illustrated in FIGS. 6 and 7, guide wire 24, initially introduced into the left femoral artery (not shown), extends to heart 98 through descending aorta 100, over aortic arch 102, down into ascending aorta 104, through aortic valve 106, and into left ventricle 108. Thus, guide wire 24 provides a guide for advancing catheter 10 and positioning the distal end 28 of secondary catheter 26 within the body of the subject.

Once the guide wire is positioned within the body, the catheter is engaged with the guide wire via the guide wire lumen and advanced along the guide wire to the target position or structure. In the illustrated embodiment, catheter 10 is shown engaged with guide wire 24 and inserted into left ventricle 106 of heart 98 by the same pathway through the vasculature as guide wire 24—through descending aorta 100, over aortic arch 102, down into ascending aorta 104, through aortic valve 106, and into left ventricle 108.

The secondary catheter may be carried along with the catheter, or the secondary catheter may be introduced into the catheter, once the catheter is in place, and advanced through the delivery lumen to a position within the body. In the illustrated embodiment, the distal end 28 of secondary catheter 26 is shown extending through side port 16 into left ventricle 106 adjacent endomyocardial septum 110.

Similar to positioning the guide wire, positioning the catheter and/or secondary catheter may accomplished by image enhancement. For example, the catheter or secondary catheter may contain radioopaque markers to aid in imaging, or may contain a visualization device, such as a video camera, for assisting the operator in determining the position of the catheter and/or secondary catheter within the body.

In some embodiments, the target structure is a certain portion of the heart, such as a ventricular septum. In specific embodiments, the target structure is the endomyocardial septum below the aortic or mitral valve. For example, as illustrated in FIGS. 6 and 7, guide wire 24, catheter 10, and secondary catheter 26, have all been directed into left ventricle 106 of heart 98. The distal end 28 of secondary catheter 26 is positioned adjacent to superior endomyocardial septum 110 below aortic valve 108 and mitral valve 112. As illustrated, the distal end 28 of secondary catheter 26 can be positioned adjacent endomyocardial septum 110 while both catheters 10, 26 remain in the superior portion of left ventricle 106. Thus, it is possible to use this device to ablate tissue of the endomyocardial septum beneath the aortic valve without introducing any portion of the catheter or secondary catheter into the inferior portion of the left ventricle. In fact, while guide wire 24 is shown positioned within the inferior portion of left ventricle 106 in FIGS. 6 and 7, with the distal end 42 of guide wire 24 positioned adjacent the base of left ventricle 106, it is not necessary for guide wire 24 to extend so far into ventricle 106. Instead, the distal end of the entire device—including the guide wire—could remain within the superior half of the left ventricle while still allowing the distal end of the secondary catheter to be positioned adjacent the endomyocardial septum inferior to the aortic valve.

The device may be used in a variety of diagnostic and treatment methods. In some embodiments, the secondary catheter includes a pick or boring mechanism for puncturing or cutting channels through tissue or occlusions. For example, the catheter may be used to create channels in tissue to aid in blood flow. In other embodiments, the secondary catheter contains a mechanism for collecting a tissue sample, such as biopsy forceps, that allows the device to be used to biopsy tissue, such as from the interior wall of the heart. In yet other embodiments, the secondary catheter includes a hollow tube and needle used to deliver some solid or liquid agent, such as a drug or recombinant nucleic acid, to a desired location within the body, such as to the wall of the heart.

In some embodiments, the secondary catheter contains means for ablating tissue at its distal end, as described above. In such embodiments, the device may be used to ablate tissue from a particular organ or structure within the body, including ablating tissue of the endomyocardial septum 110 as illustrated in FIGS. 6 and 7. In particular embodiments, the device is used to ablate tissue in a subject having a particular disease. For example, because the side port is on the side of the catheter, the secondary catheter can be directed substantially perpendicularly out of the side of the catheter and into the endomyocardial septum below the aortic valve; thus, the device may be used to treat a subject having septal hypertrophy, including hypertrophic cardiomyopathy, by ablating tissue in the aortic outflow tract of the subject. Additionally, the catheter could be similarly used in other chambers of the heart, for example in the right ventricle to treat areas below the pulmonary artery valve to perform infindibular ablation for the treatment of pulmonic stenosis. However, the apparatus and methods described herein are useful in procedures other than endomyocardial septal ablation or treatments for hypertrophic cardiomyopathy, such as procedures where a delivery device needs to be extended through a flexible cathether to other points within the body.

In one specific embodiment, hypertrophic cardiomyopathy is treated using the device illustrated in FIGS. 3, 6-7 and described above. Guide wire 24 is introduced percutaneously into an artery (not shown) of a subject and advanced through the vasculature into left ventricle 106 of a subject's heart 98. Guide wire 24 is inserted into guide wire lumen 12 of flexible catheter 10, and guide wire lumen 12 is advanced along guide wire 24 until side port 16 advances through the aortic valve 108 and is substantially aligned with a hypertrophic septal myocardium. In the particular illustrated embodiments, this hypertrophic septal myocardium is the superior endomyocardial septum 110, almost immediately below aortic valve 108.

Once side port 16 is in place, a therapeutic or diagnostic agent is introduced through delivery lumen 14 and side port 16, such that side port 16 directs the agent at the hypertrophic septal myocardium, such as the endomyocardial septum 110. In some embodiments, the therapeutic or diagnostic agent is introduced through delivery lumen 14 and side port 16 via secondary catheter 26, which is inserted into delivery lumen 14 before or after guide wire 24 is inserted into guide wire lumen 12. In such embodiments, secondary catheter 26 is directed substantially transverse to the longitudinal axis of flexible catheter 10. Secondary catheter 26 may include any ablative device or means for delivering a diagnostic or therapeutic agents as described above, such as the illustrated needle 34. In particular embodiments, secondary catheter includes a radiant energy delivery probe, such as a thermal, laser, or radiofrequency delivery probe.

In some embodiments, the therapeutic or diagnostic agent is an ablative agent and is administered at the site of the enlarged heart tissue. The amount of agent and/or the length of time of administration may depend on the degree of enlargement of heart tissue, type of ablative agent used, physiological characteristics and health of the subject, and other considerations. In any case, the ablative agent is administered in an amount and length of time sufficient to reduce the thickness of the myocardium and improve the rate of blood flow through the aortic outflow tract. For example, if laser energy is used as the ablative agent, a laser producing about 50 to 60 mJ/mm² directed at an area of the endomyocardial septum measuring 100 mm² for a time period of about 3 to 5 seconds can effectively reduce the thickness of the myocardium and measurably improve blood flow through the aortic outflow tract. As another example, infusion of about 1-2 mL ethanol into a 1000 mm³ volume adjacent the endomyocardial septum can ablate an amount of tissue sufficient to reduce the thickness of the myocardium and measurably improve blood flow through the aortic outflow tract. Ablation also may be accomplished by multiple such ethanol injections. Additionally, in certain embodiments, multiple injections of one or more agents are used to achieve a desired therapeutic effect or to monitor the effects of ablation. For example, multiple injections of an ablative agent and a contrast agent may be used to monitor the reduction in the outflow obstruction associated with hypertrophic cardiomyopathy, or may be used to monitor velocity or pressure of blood flowing by the superior endomyocardial septum.

Having illustrated and described the principals of the invention by several embodiments, it should be apparent that those embodiments can be modified in arrangement and detail without departing from the principals of the invention. Thus, the invention as claimed includes all such embodiments and variations thereof, and their equivalence, as come within the true spirit and scope of the claims stated below.

I claim:

1. A device for delivering a therapeutic or diagnostic agent to a heart, comprising:
a flexible catheter having a proximal end and a distal end, a guide wire lumen that extends longitudinally through the catheter, and a delivery lumen that extends longitudinally at least partially through the catheter and communicates with a side port adjacent a distal end of the catheter, through which side port the agent is to be delivered.

2. The device of claim 1, wherein the delivery lumen includes a deflection member that connects the longitudinally extending delivery lumen with the side port.

3. The device of claim 2, wherein the deflection member comprises an intermediate curved wall at a terminal end of the delivery lumen that connects the delivery lumen to the side port.

4. The device of claim 1, wherein the guide wire lumen and delivery lumen extend substantially parallel to one another through the catheter.

5. The device of claim 3, wherein the curved wall changes a direction in which the delivery lumen extends, from longitudinally through the catheter to substantially transverse, such that the side port opens through a side wall of the catheter.

6. The device of claim 1, wherein the catheter is a steerable catheter.

7. The device of claim 1, further comprising a guide wire along which the guide wire lumen is capable of sliding to guide the catheter into a desired anatomic position within the heart.

8. The device of claim 1, further comprising a secondary catheter capable of sliding through the delivery lumen.

9. The device of claim 8, wherein the secondary catheter comprises a catheter capable of delivering a liquid agent, a solid agent, or radiant agent to the heart.

10. The device of claim 9, wherein the secondary catheter comprises a radiofrequency probe.

11. A device for delivering a therapeutic or diagnostic agent to a heart, comprising:
an elongated flexible catheter having a proximal end and a distal end, a guide wire lumen that extends longitudinally completely through the catheter to form guide wire ports in each end of the catheter, and a delivery lumen that extends longitudinally through the catheter substantially parallel to the guide wire lumen and communicates with a side port adjacent a distal end of the catheter, through which side port the agent is to be delivered, wherein the delivery lumen curves from extending longitudinally to extending substantially transversely to communicate with the side port.

12. A kit, comprising:
the catheter of claim 11; and
a guide wire along which the guide wire lumen can slide.

13. The kit of claim 12, further comprising an agent delivery device for introducing a therapeutic or diagnostic agent through the delivery lumen into the heart.

14. The kit of claim 13, wherein the agent delivery device comprises a flexible secondary catheter.

15. The kit of claim 14, wherein the secondary catheter comprises an energy delivery probe.

16. The kit of claim 15, wherein the energy delivery probe is a radiofrequency, mechanical, or thermal energy delivery probe.

17. The kit of claim 13, further comprising instructions for advancing the catheter along the guide wire into a ventricle of the heart with the side port adjacent a ventricular septum of the heart, then delivering the therapeutic or diagnostic agent through the delivery lumen and side port to the ventricular septum.

18. An assembly for delivering therapeutic or diagnostic agents to the heart, comprising:
an elongated flexible catheter having a proximal end and a distal end, a delivery lumen extending from the proximal end to at least adjacent the distal end of the catheter, a side port adjacent the distal end which communicates with the delivery lumen, and a guide wire lumen extending through the proximal end and through the distal end;
a guide wire through the guide wire lumen, along which the flexible catheter slides; and
a delivery catheter that extends through the delivery lumen.

19. The assembly of claim 18, wherein the delivery lumen and guide wire lumen are separate from and extend substantially parallel to one another through the catheter.

20. The assembly of claim 18, wherein the delivery catheter comprises a radiofrequency or thermal energy delivery probe.

21. The assembly of claim 18, wherein the delivery lumen curves from a longitudinal to a transverse pathway in the elongated flexible catheter to form a curved wall that directs the delivery catheter toward the side port.

22. A method of delivering a therapeutic or diagnostic agent to the endocardium of a heart, comprising:
providing an elongated flexible catheter having a proximal end and a distal end, a delivery lumen extending from the proximal end to at least adjacent the distal end of the catheter, and a side port adjacent the distal end which communicates with the delivery lumen;
advancing the flexible into a chamber of the heart, until the side port is substantially aligned with a target structure in the chamber of the heart; and
introducing the therapeutic or diagnostic agent through the delivery lumen and side port at the endocardium of the heart.

23. The method of claim 22, wherein the flexible catheter further comprises a guide wire lumen extending through the proximal end and the distal end of the catheter, and wherein advancing the flexible catheter into the chamber of the heart comprises advancing the guide wire lumen along an intra-vascular guide wire which extends into the chamber of the heart.

24. The method of claim 23, further comprising introducing the guide wire percutaneously into a blood vessel and then into the chamber of the heart.

25. The method of claim 23, wherein the chamber of the heart is a ventricle.

26. The method of claim 23, wherein the target structure is a ventricular septum.

27. The method of claim 23, wherein advancing the guide wire lumen along the guide wire comprises advancing the flexible catheter until the side port is positioned in the ventricle below the aortic or mitral valve.

28. The method of claim 27, wherein the target structure in an endomyocardial septum.

29. The method of claim 23, wherein the method is a method of ablating tissue in an aortic outflow tract of a subject who has septal hypertrophy.

30. The method of claim 29, wherein the method is a method of ablating tissue in an aortic outflow tract of a subject who has hypertrophic cardiomyopathy.

31. The method of claim 23, wherein the introducing the agent through the delivery lumen comprises introducing an ablative agent.

32. The method of claim 31, wherein introducing the ablative agent comprises introducing a pharmaceutical, chemical, biological, mechanical, or radiant energy agent.

33. The method of claim 32, wherein introducing the radiant energy agent comprises introducing thermal or radiofrequency energy through the side port into the endocardium.

34. A method of treating hypertrophic cardiomyopathy in a subject, comprising:
providing an elongated flexible catheter having a proximal end and a distal end, a delivery lumen extending from the proximal end to at least adjacent the distal end of the flexible catheter, a side port adjacent the distal end which communicates with the delivery lumen along a shoulder portion that curves from a longitudinal to a substantially transverse direction within the flexible catheter, and a guide wire lumen extending separate from and parallel to the delivery lumen, wherein the guide wire lumen extends through the proximal end and through the distal end of the flexible catheter;
introducing a guide wire percutaneously into an artery of the subject, and advancing the guide wire into a left ventricle of the heart of the subject;
advancing the guide wire lumen of the flexible catheter along the guide wire, until the side port advances through an aortic valve, and the side port is substantially aligned with hypertrophic septal myocardium; and
introducing a therapeutic agent through the delivery lumen and side port, such that the side port directs the agent at the hypertrophic septal myocardium.

35. The method of claim 34, wherein introducing a therapeutic agent through the delivery lumen and side port comprises introducing a secondary catheter through the delivery lumen, such that the secondary catheter is directed substantially transversely with respect to the flexible catheter.

36. The method of claim 35, wherein introducing a secondary catheter through the delivery lumen comprises introducing a radiant energy delivery probe.

37. The method of claim 36, wherein the radiant energy delivery probe is a thermal or radiofrequency delivery probe.
